(19) United States
(12) Patent Application Publication
(10) Pub. No.: US 2010/0010546 A1
(43) Pub. Date: Jan. 14, 2010

(54) Minimally Invasive Instruments and Methods for the Micro Endoscopic Application of Spine Stabilizers in the Interspinous Space

(76) Inventor: Elias Humberto Hermida Ochoa, Cuauhtemoc (MX)

Correspondence Address: MILLER, MATTHIAS & HULL, ONE NORTH FRANKLIN STREET, SUITE 2350, CHICAGO, IL, 60606, US

(21) Appl. No.: 12/171,988

(22) Filed: Jul. 11, 2008

(51) Int. Cl. A61B 17/88

(52) U.S. Cl. 606/279; 606/86 A

(57) ABSTRACT

The present disclosure provides a safe and simple method, as well as a new instrumentation to improve the technique of the minimally invasive spine surgery through micro endoscopy with the use of hooks and a new improved stabilization interspinous device or implant which is placed as an auxiliary in the function of the affected intervertebral disk, by relieving the pressure on spinal nerves.

### FIELD OF THE DISCLOSURE

The present disclosure generally relates to a method and instrumental for the positioning of stabilization interspinous devices through a minimally invasive surgery and, more particularly, to the use of a novel instrumental of hooks for the positioning of stabilization interspinous devices through a minimally invasive surgery for the micro endoscopic application.

### BACKGROUND OF THE DISCLOSURE

The degenerative disk disease (DDD) of spine is a relatively common procedure among elderly people; however, it may be present in people at any age. Even though it is a normal part of aging, the degenerative discopathy may cause pain in the back and neck and other symptoms.

With age, intervertebral disks lose flexibility, elasticity and absorption capacity of impact. At the same time, the pulpy core starts to dehydrate. As a consequence, people suffer from pain and stiffness in the back.

In most people, these changes become present in a gradual way and the treatments for such suffering may be non-surgical treatments which comprise analgesic, anti-inflammatory and muscle relaxant, physiotherapy and practice of stretching exercises to improve flexibility, heat/cold-based therapy, and surgical treatments which are generally used when discomfort is persistent even after having tried non-surgical treatments that generally comprise the use of stabilizer implants or vertebral spacers.

The traditional surgical method of vertebral decompression is performed through the methods of laminectomy or laminotomy, which generally require two or three hospitalization days after the surgery. Among the optical instruments used in these procedures, microscope, endoscope and magnifying glasses could be mentioned. The retractor of the tissue commonly used in these kinds of surgeries usually comprises two blades and recently retractors have been used with tubular shape.

Some surgical solutions of the prior art are based on the fusion of adjacent vertebras through the positioning of a bridge or immobilizer fixed to the bone from a vertebra on and a vertebra under the affected disk. The surfaces of the fusion of the bone may include the rear vertebral elements, the vertebral platforms or a combination of both. Sometimes a positioning of metallic instrumentation is used to stabilize the fusion of the vertebral segment.

Due to the substantially invasive nature of traditional surgery for these kinds of pathologies, in many cases, many hours in the operating room are required and sometimes even weeks for a post-operative recovery due to excessive manipulation of the tissue during the surgical procedure.

The status of the technique related to the present disclosure may be found in: European Patent No. EP880938A1 “Instrumentation for implant insertion”; US Application No. US20050228380A1 “Instruments and methods for minimally invasive spine surgery”; US Application No. US2008045957 “Spinal Stabilization Systems and Methods Using Minimally Invasive Surgical Procedures”; U.S. Pat. No. 5,891,147 “Minimally invasive spinal surgical methods & instruments”; U.S. Pat. No. 5,792,044 “Devices and methods for percutaneous surgery”, US Patent Application US2005/0261768A1 “INTERSPINOUS SPACER”.

In the document of U.S. Pat. No. 5,792,055 published by inventors Kevin T. Foley et al. the steps of a method to access a surgical site in the spine are described (column 10 from line 11 to column 16 line 9 and through FIG. 10a to FIG. 10i). According to what is described in such patent, the surgery starts with the insertion of a guide wire followed by a series of dilators bigger each time used sequentially to distend soft tissues until they get to the largest dilator with the required diameter. After that, a cannula or retractor is introduced and it moves forward on the biggest dilator to provide a working channel from the patient's skin to the adjacent working space to the spine. The retractor may be fastened in the place through any of the many mentioned means in such patent already known in the art.

It is convenient to use the working channel provided by the retractor for the surgical tools, devices of vision and for the insertion elements and manipulation of fixation, to the greatest possible extent for the desirable positioning and fixing.

Sometimes it is convenient to have a working space in the spine wider axially than transversally. However, to provide such access with the use of common circular retractors and dilatation techniques above mentioned, such a big diameter may be require to generate a significant trauma to tissues involved during the positioning of the dilator and tubular retractor. The development of minimally invasive percutaneous procedures has caused an important improvement in the reduction of recovery time and post operative pain due to the requirement of minimal dissection of tissue (for example: skin and muscular tissue). The minimally invasive surgical techniques are desirable for spine and neurosurgical applications due to the need to access the inner part of the body and that risk of harm to vital tissues involved decreases.

In the document US Patent Application Publication No. US2005/0261768A1, a method to place an interspinous spacer among adjacent vertebras and several spacer models is disclosed. However, those devices do not present the same volumetric disposition and do not count on fixation sutures or retention devices to assure the implant, so that there is not a strong or reliable implant since it may move with a flection effort of the trunk.

The method and instrumental of the present disclosure allow to perform the fixation of any implant (for example: DIAM® (Device for Intervertebral Assisted Motion or also known as Dynamic Intervertebral Assisted Motion), modifying the disposition of the buttonholes (19) from the sutures, so that both sutures (17) are fixed to the same side of the implant DIAM® through the use of hooks claimed herein, in which damages are minimized in the adjacent tissues.

In the document of the prior art “Device for intervertebral assisted motion: technique and initial results” published by JEAN TAYLOR, M.D., PATRICK RUPIN, M.D., STEPHANE DELAJOUX, M.D., and SYLVAIN PALMER, M.D., regarding the implant DIAM®, the steps for its positioning are mentioned.

The instruments used in the procedures are showed in FIG. 1 of such document, the technique is shown in FIG. 2 and the post-operative image projection selected may be observed in FIG. 3. In the bilateral procedure, which sacrifices ligaments, after termination of any necessary decompression, the supraspinous and interspinal ligaments are dissected for the proper application of the implant DIAM®. A space is made with the chisels and, afterwards, such space is distended and test measures are taken to determine the size of the implant DIAM® that will be used. The implant is pressed through a clamp to reduce its size and allow the insertion with the aid of such clamp and a mallet. The sutures of the implant surround the spinal apophysis above and under the implant DIAM®, and are fastened and afterwards, they are fitted with bushings. The wound is then irrigated and closed in a standard way. Post-operative immobilization is not required.

The progress in minimally invasive surgery represents a significant advance; nevertheless, it is still a need to develop additional minimally invasive surgical instruments and methods causing the least damage and subsequently with less recovery time.

The method and instrumental herein disclosed reduce, considerably, the surgery room time, costs of surgical procedure, tissue damage in patients and provide a quicker recovery than up to day known techniques.

There are several kinds of stabilization interspinal devices, such as implants placed among adjacent vertebras to substitute the function of the affected inter vertebral disk. A known example is the implant DIAM®, which was created by JEAN TAYLOR of Clinique de L'Esperance in Nice.

The method of rear dynamic interspinal stabilization through a balance device or implant DIAM®, helps dissipate the charge of the disk, restores tension in the rear band aligning the line of joint facets and increases foraminal height. The implant DIAM® has a silicone core with a coating of mesh and ties made of polyester. Its butterfly shape (as one of the embodiments or presentation or form) suitably adapts to the space between the two adjacent spinal apophysis to the damaged disk. The device is available in a variety of sizes between 8 and 14 mm, to allow the surgeon to place the most suitable for the anatomy of the patient.

The method implies removing the interspinal ligament without damaging the supra spinal ligament, and the surgeon places the respective implant in the space generated by dissecting the interspinal ligament.

By positioning the device DIAM®, pain is reduced, since the support is improved and the friction is reduced due to the loss of volume or elasticity of the damaged disk.

The operation, is a relatively simple surgery, not very invasive (the incision on the skin is about 30 mm) and does not require the surgeon to operate within the vertebral channel, therefore, the neurological commitment risks are minimal, see the following bibliographical references:

a) Device for intervertebral assisted motion: technique and initial results, Neurosurg. Focus/Volume 22/January, 2007.

b) Posterior Dynamic Stabilization using the DIAM (Device for Intervertebral Assisted Motion). Jean Taylor. MD. Centre Hospitalier Princesse Grâce—MONACO.

c) Patient Information Brochure, Medtronic Sofamor danek Autralasia, 4/446 Victoria Road, Gladesville, NSW 2111.

d) Minimally Invasive Dynamic Stabilization of the Lumbar Motion Segment with an Interspinous Implant, Author: J. Sénégas, Minimally invasive Spine Surgery; pp 459-465.

The techniques disclosed in such documents present several disadvantages, since tissue cuttings have not been reduced effectively, therefore, post-operative pain is persistent, blood loss, longer recovery and hospital staying, due to the big incision that has to be made to place the implant.

### SUMMARY OF THE DISCLOSURE

The present disclosure provides a fast, safety and simple method, as well as a new instrumentation to improve the spine minimally invasive surgery technique with the use of hooks and a new improved interspinous stabilization device, which is positioned in the place of the interspinous ligament of the affected spinal zone to offer support and lighten pressure on spinal nerves.

An object of the present disclosure is to maintain integrity of spinal channel by performing an ambulatory surgery with minimal cuttings in tissues to achieve a quick recovery, reducing costs and time in the surgery room.

Another object of the present disclosure is to avoid fibrosis around the spinal nerves to avoid an open exposure.

Another object of the present disclosure is to provide hooks as new instrumental.

Another object of the present disclosure is to provide an interspinous device of improved stabilization.

Another object of the present disclosure is to provide a method to carry out fixation L5-S1 in patients presenting a spinal apophysis in S1.

Another object of the present disclosure is to provide a method to carry out fixation L5-S1 in patients who do not present a spinal apophysis in S1.

All the previous features and advantages and others of the disclosure will be better understood through the following illustrative description but not limiting the preferred embodiments of the same, with reference to the attached drawings.

### BRIEF DESCRIPTION OF THE DRAWINGS

FIG. 1 shows a design of a hook used to perform the previously referred method, as well as its structural features and parts.

FIG. 2 shows a perspective view of dilators used to make the access diameter bigger of the micro endoscopy system.

FIG. 3 shows the micro endoscope X-Tube® placed in a proper position for the surgical intervention through a retention arm in the desired position and the curve Kerrison clamp.

FIG. 4 shows the chisels to create the access space of the suture around the spinal apophysis.

FIG. 5 shows the use of measurer in the interspinous space to determine the size of the implant DIAM® that will be used.

FIG. 6 presents a perspective view of a modification in the sutures of the implant DIAM®.

FIG. 7 shows the use of the hook to make the suture pass around one of the spinal apophysis involved in the procedure.

FIG. 8 shows the sutures once placed around the spinous apophysis involved.

FIG. 9 presents the implant DIAM® with the sutures which have passed through the buttonholes before its insertion.

In FIG. 10, the way the sutures pass through the buttonholes is observed in detail.

FIG. 11 shows the Peang clamps which may be used for the insertion of an implant DIAM® in the interspinous space through a claimed method.

FIG. 12 presents a view of the micro endoscope on the opposite side in which the positioning of the sutures around the spinous apophysis is observed.

In FIG. 13 an aerial view of the implant DIAM® with the sutures suitably placed around and through the buttonholes and ready for the insertion is observed.

FIG. 14 shows the implant DIAM® which has been deformed through the use of Peang clamps and positioned in the interspinous space.

FIG. 15 shows the implant DIAM® within the intervertebral space.

FIG. 16 shows the rear view of the implant DIAM® within the intervertebral space.

FIG. 17 shows the way to adjust the sutures according the implant DIAM® is introduced to avoid the sutures mixing and obstructing the passing of the implant.

FIG. 18 shows the shape the implant takes when the sutures are fastened to offer the proper support and avoid displacement of said implant DIAM®.

In FIG. 19 the use of the mallet employed as an aid to fit the implant and fix it securely in the proper position for its safety settling.

FIG. 20 shows the bushing, which may be made of titanium or any other bio-compatible material used to fix the sutures once they are fastened and avoid they loosen or unfasten.

FIG. 21 shows how to hold the bushing and move it through the suture until being in contact with the implant and fasten the bushing by the deformation of the cylindrical body due to the pressure of the strapping clamps.

FIG. 22 presents a lateral view of the implant DIAM® properly placed and fastened.

### DETAILED DESCRIPTION

Regarding FIG. 1, a hook in distal support with a length of about 21 cm is observed, it has a retention sleeve (1) with a knurled surface which offers a nonskid feature, followed by a rod (2) continuing in a curvature (3) of 60±15 degrees regarding the axis of the rod and arch length (4) of about 50±10 mm, the hook which in its distal end has a fishhook-shaped tip (5) with an opening of about 8 degrees, which allows to fasten the suture of any implant. The curvature and arch length of the hook used in the method disclosed herein will be determined by the size of the involved spinous apophysis.

The stages of preparation previous to the method for the micro endoscopy surgery disclosed herein, are the ones already known in the art. In the claimed method the steps to locate the zone of the affected central intervertebral space (16) are involved, identifying the upper spinous apophysis (15) and the lower spinous apophysis (14) and marking the insertion site with a line parallel to about 30 mm of the medium line of the spine; indicating the insertion site with a Steinmann nail; making an incision on the skin of about 24 to 26 mm; introducing dilators (see FIG. 2) from small to big diameters through the incision until it reaches the biggest diameter by which a controlled opening tube is introduced, X-tube® (13) of Medtronic® (see FIG. 3), where the diameters of such dilators increase gradually (6) until it reaches the largest diameter (7) to allow the entrance to device X-Tube® (8) in its non expanded configuration whose diameter of entrance is 26 mm which may expand the percutaneous working space without implying performing a bigger incision al the moment of positioning such micro endoscopy; once the X-Tube® is placed, the part of the distal end expands to its maximum capacity which is 40 mm and it addresses to the central interspinous space (16), thus getting a wider working space than with other existent micro endoscopy systems in the market; the retention arm (9) is fixed which includes the X-Tube®; the frontal part of the interspinous ligament is dissected in the central interspinous space (16), respecting the rear part to make the excursion of the suture easier through the use of curve Kerrison clamps (10) preserving the supraspinal ligament (24), which offers support and stability to the affected vertebras and allows more stability and less complications in the recovery. For this procedure any instrument selected of microscopes or magnifying glasses as a visual aid is used to carry out the surgery.

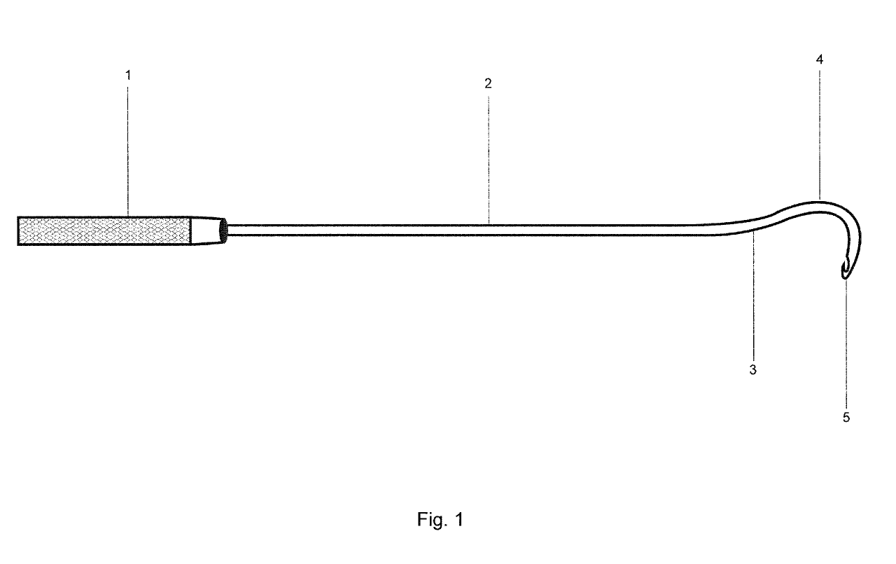

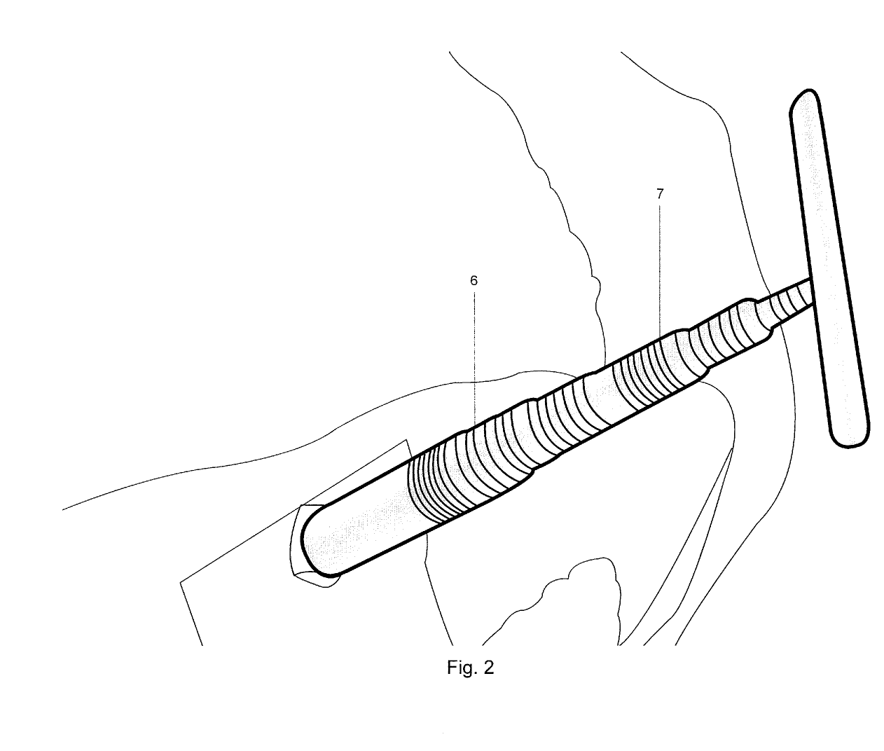

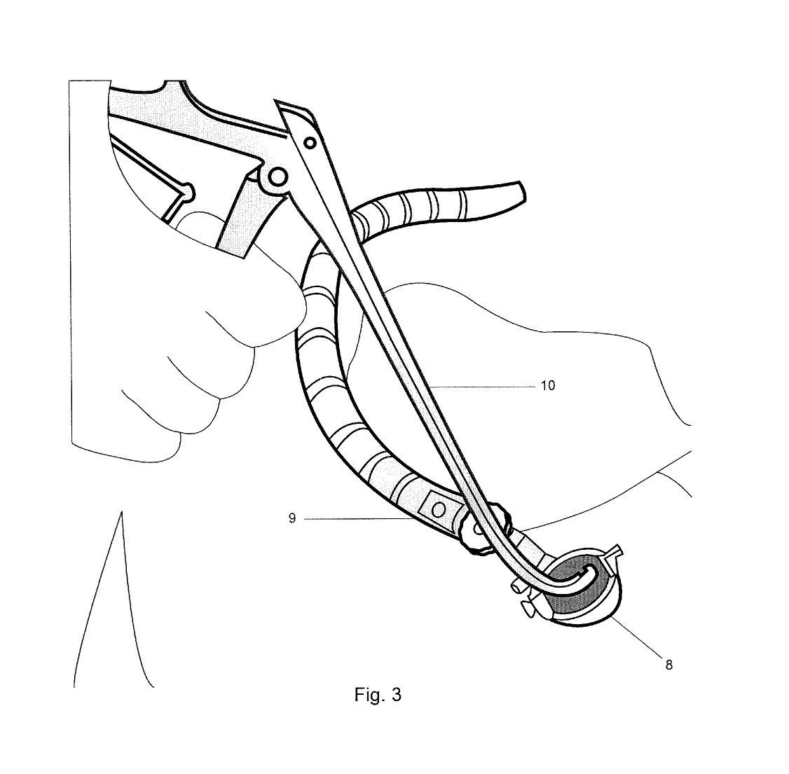

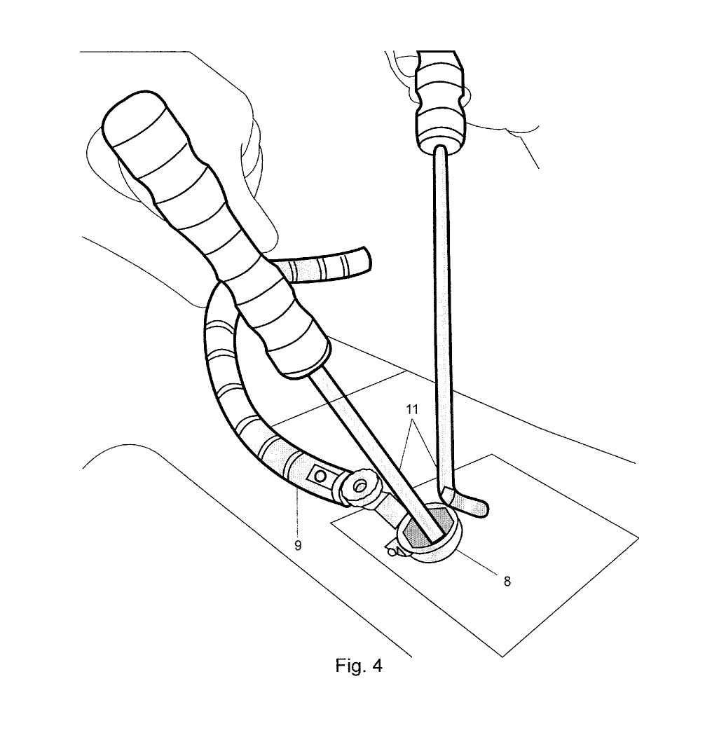

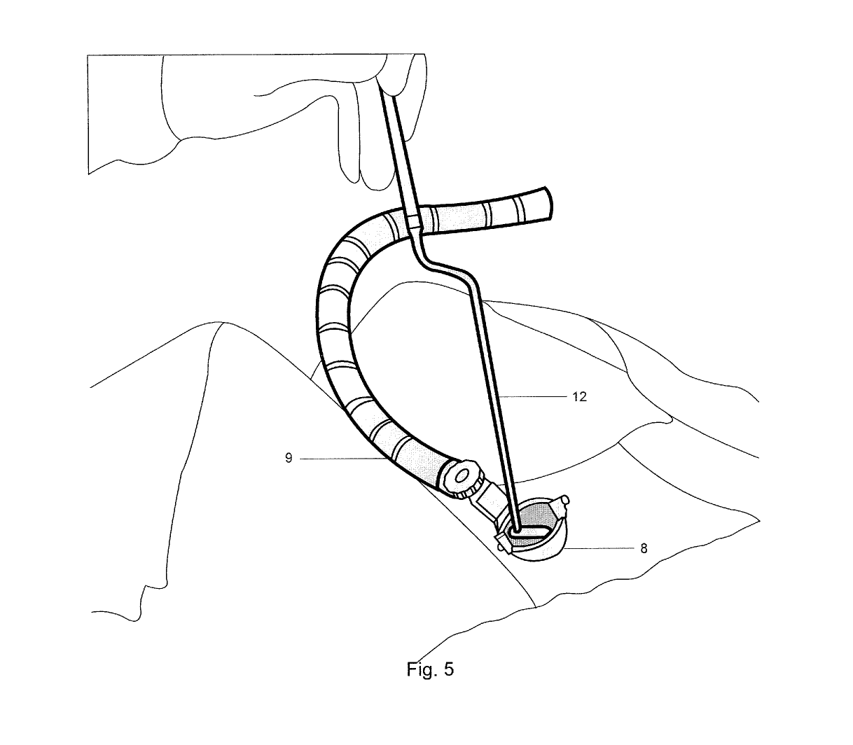

Once the interspinous ligament contained among the vertebral disks involved is removed, then the chisels (11) are used which have a spin with the end of the cutting with a curvature likewise and the other one presents a curvature in the end of the cutting on the opposite direction (FIG. 4), which are used to create a channel through which a fishhook-shaped tip hook may be introduced (5) to slide the sutures around the spinous apophysis (14) and (15). This is achieved through the introduction of a thread with a likewise curvature to relief the space of one of the sides of every vertebra involved and repeating the operation with a thread with curvature on the opposite direction to free the rest of the suture path.

The instrumental used until this step of the method is the common one used for spine surgeries known in the previous art. Due to the considerably reduced space in a micro endoscopic minimally invasive surgery, the distractor, as well as the clamps used in Jean Taylor document “Device for intervertebral assisted motion: technique and initial results”, cannot be used to introduce and place the implant DIAM® in the interspinous space, to carry out the measuring of such space for the determination of the size of the suitably implant DIAM®, any tiny sharp clamp may be used as a distractor, for example, Peang clamps.

The next step is measuring the intervertebral space to determine the size of the implant DIAM® that will be used (FIG. 5). Unlike the conventional method for positioning the implant DIAM® through the minimally invasive method disclosed herein, it is not possible to introduce the distractor commonly used due to its size, this is why it is necessary to base on the use of sharp clamps, which substitute the function of the distractor and allow the use of the measurer (12) to determine the size of the implant DIAM® to be used.

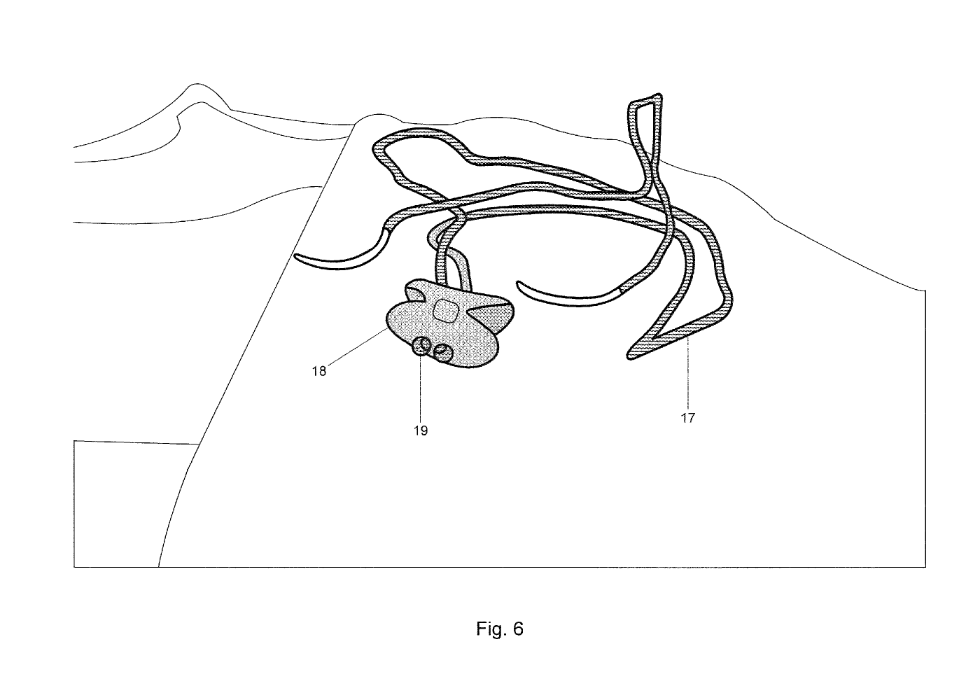

For the method herein disclosed, the location of the buttonholes (19) of the implant DIAM® has been modified to make both buttonholes to remain on the same side of the implant DIAM® as it is shown in FIG. 6.

According to FIG. 6, the new implant (18) is basically butterfly-shaped to adapt between the two spinous apophysis adjacent to the damaged disk. Such implant (18) is made of silicone with external mesh and sutures (17) made of polyester. The implant is available in a variety of sizes from 8 to 14 mm.

Such implant (18) presents, on its frontal side two buttonholes (19) adjacent and laterally opposed from each other on the rear part a pair of sutures (17) are disposed parallel corresponding among them.

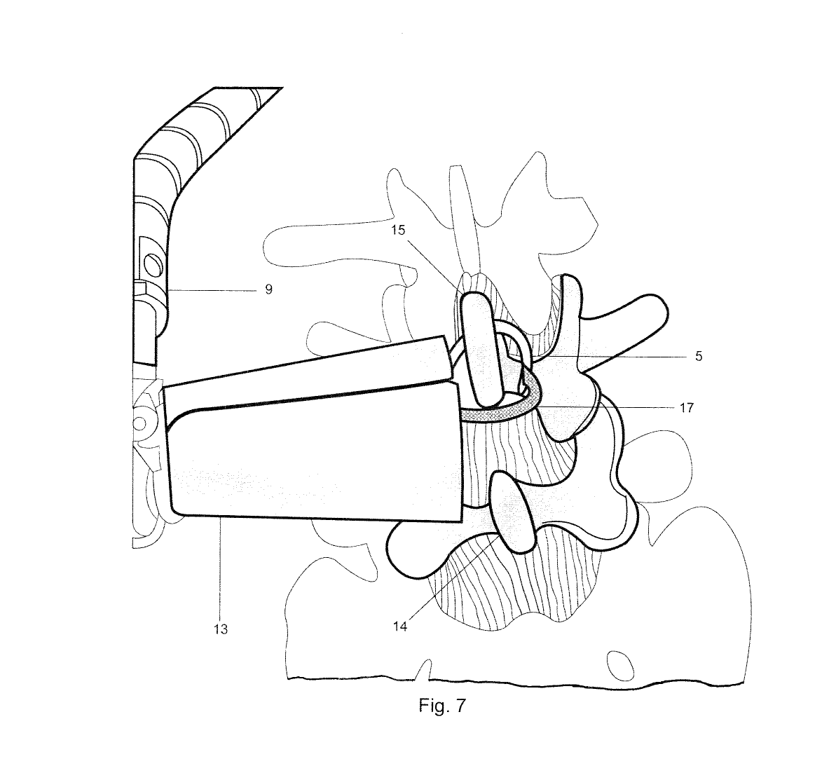

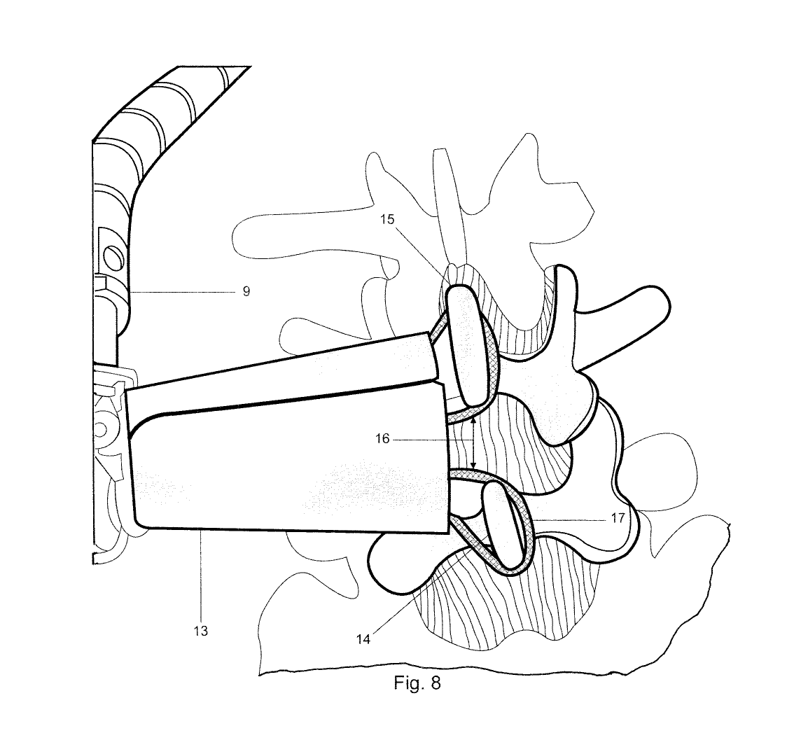

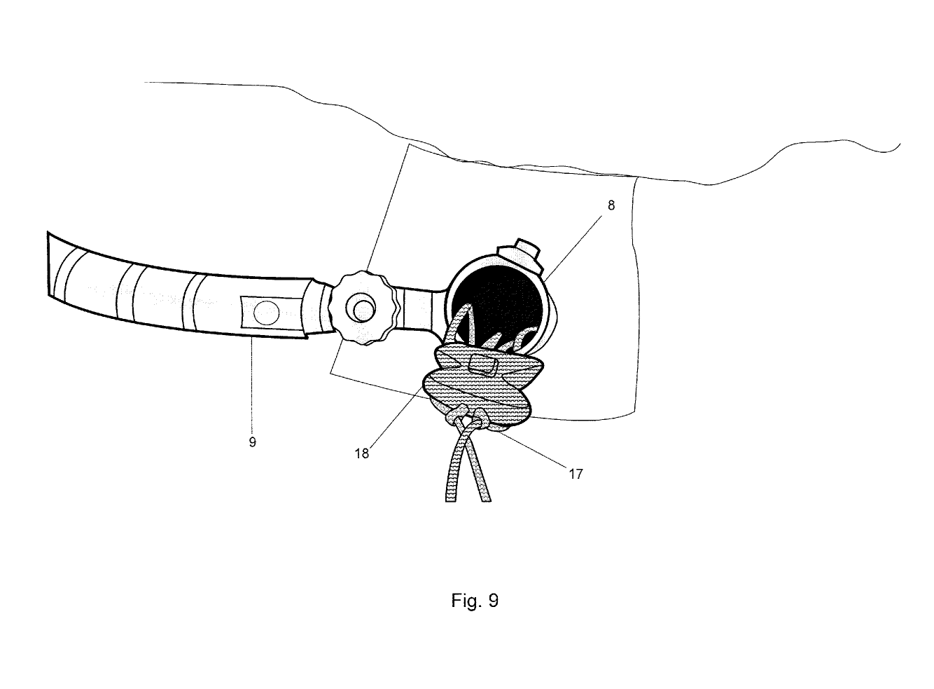

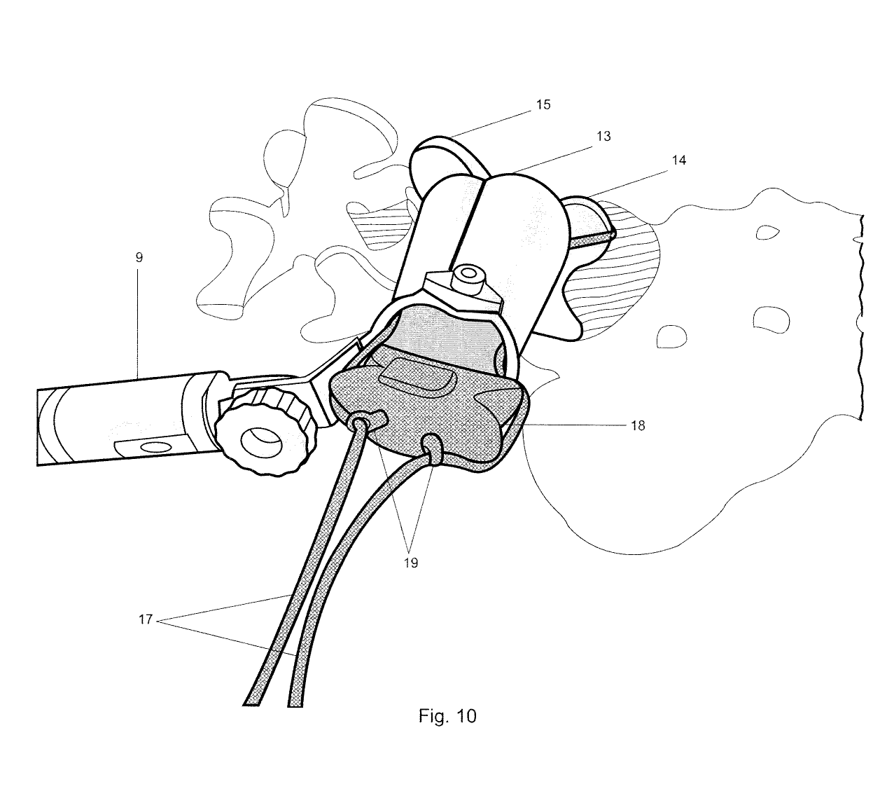

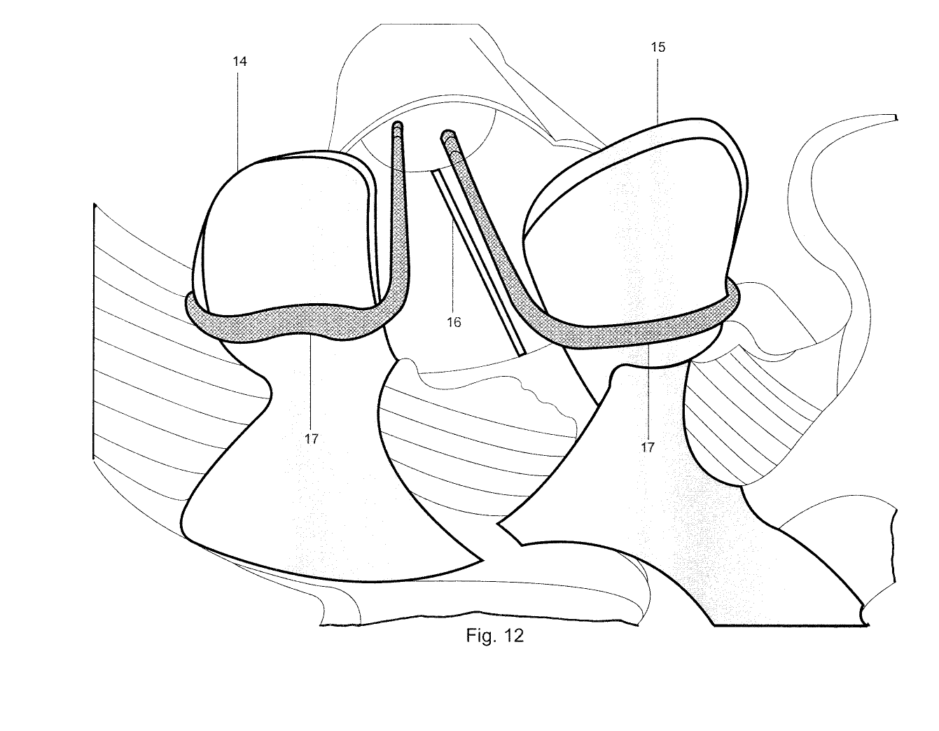

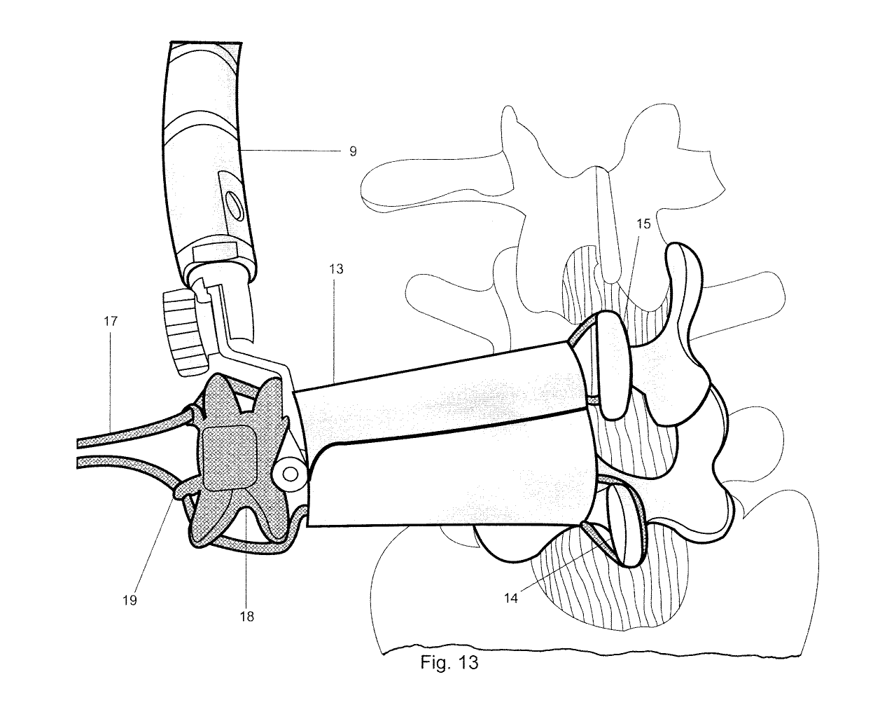

FIG. 7 shows the functioning of the instrumental of hooks. The proper hook is introduced depending on the size of the spinous apophysis, which should reach the interspinous space to achieve the retention of sutures through the channel created previously by using the chisels and it first goes toward the upper interspinous space, which is considered the adjacent space in direction to the cephalic region of the most proximal apophysis to the cephalic region, then it goes from the upper interspinous space to the central interspinous space of the site (16) where the implant will be placed (18) and the fishhook-shaped tip remains towards the front of the tube; in said fishhook-shaped tip (5) the suture (17) of the implant is anchored and it goes through the back of the upper spinous apophysis (15) and it is taken out on the front, the same operation is carried out now from the lower space which is considered the adjacent space towards the podalic region of the most proximal apophysis to the podalic region surrounding the lower spinous apophysis (14) towards the central interspinous space (16) where the implant will be, with both sutures taken to the exterior of the tube as it is shown in FIG. 12, where each suture is passed through its respective buttonhole, so that the suture surrounding the upper spinous apophysis (15) passes through the upper external part of the buttonhole of the upper side, and for the suture which slides around the lower spinous apophysis (14) the suture passes through the lower buttonhole from the bottom to the top (FIG. 9 and FIG. 10).

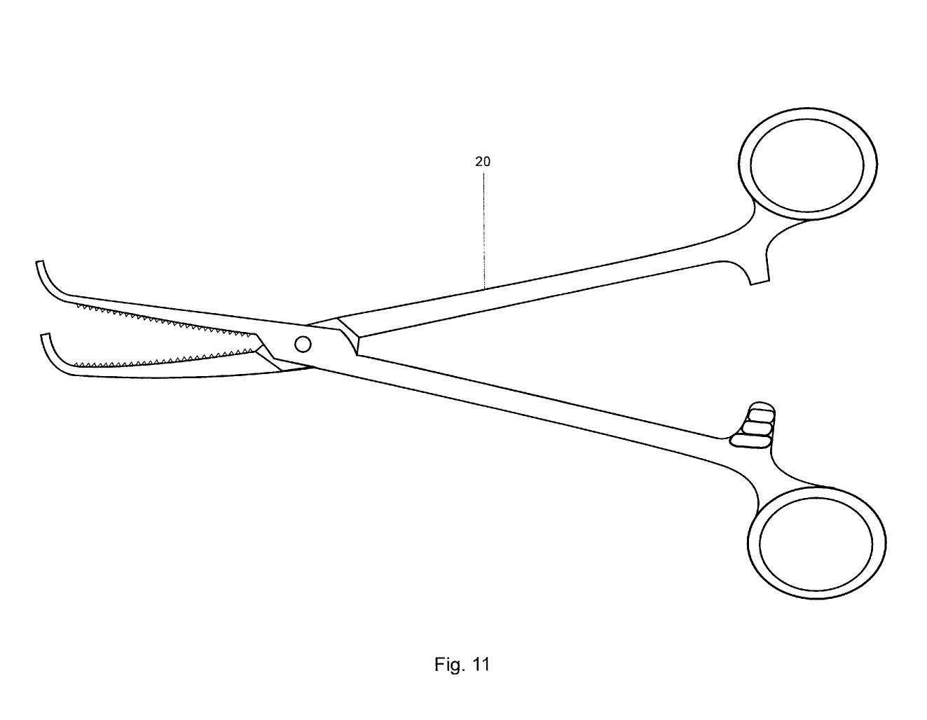

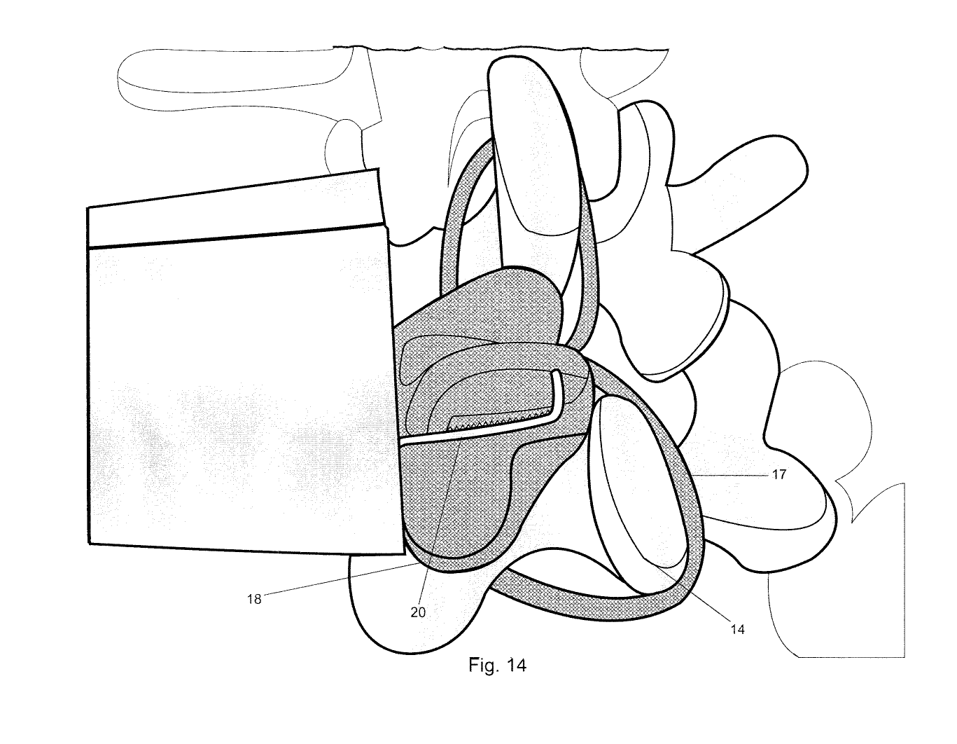

Once the retention sutures are positioned around the spinous apophysis (14) and (15) of the involved vertebras through the use of the instrumental of the claimed hooks in the present disclosure, those sutures pass through the buttonholes (19) of said implant. It is fastened with the two sutures towards the front of the tube as it is shown in FIG. 10. To introduce the implant a clamp is used to let fastening and deforming the implant laterally in the direction of the sutures in a suitable way the implant DIAM®, for example, a Peang clamp. FIG. 11 shows the Peang clamps (20) which may be used to carry out a compression that causes a proper deformation as it is shown in FIG. 14, to introduce the implant and place it in the central interspinous space (16).

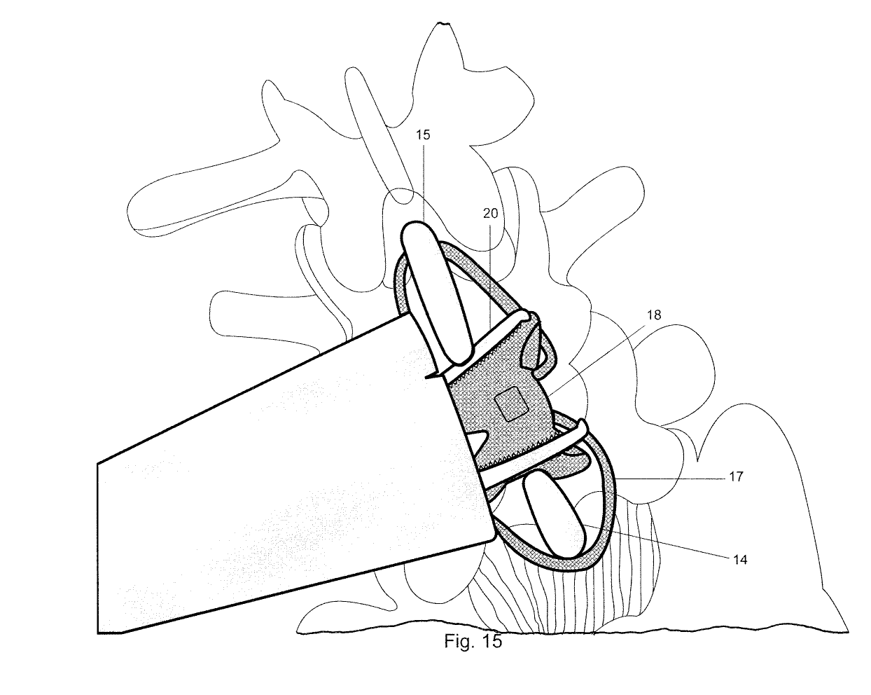

The implant DIAM® (18) is taken, in a slippery way, to the central interspinous space (16) (FIG. 14) and during that process the tension is applied in the sutures to help the displacement and, in addition, to avoid the sutures to be tied or obstruct the passing of the implant through the micro endoscope. When the implant DIAM® is in the central interspinous space (16) the clamp is freed to allow the implant DIAM® to recover its original shape (FIG. 15).

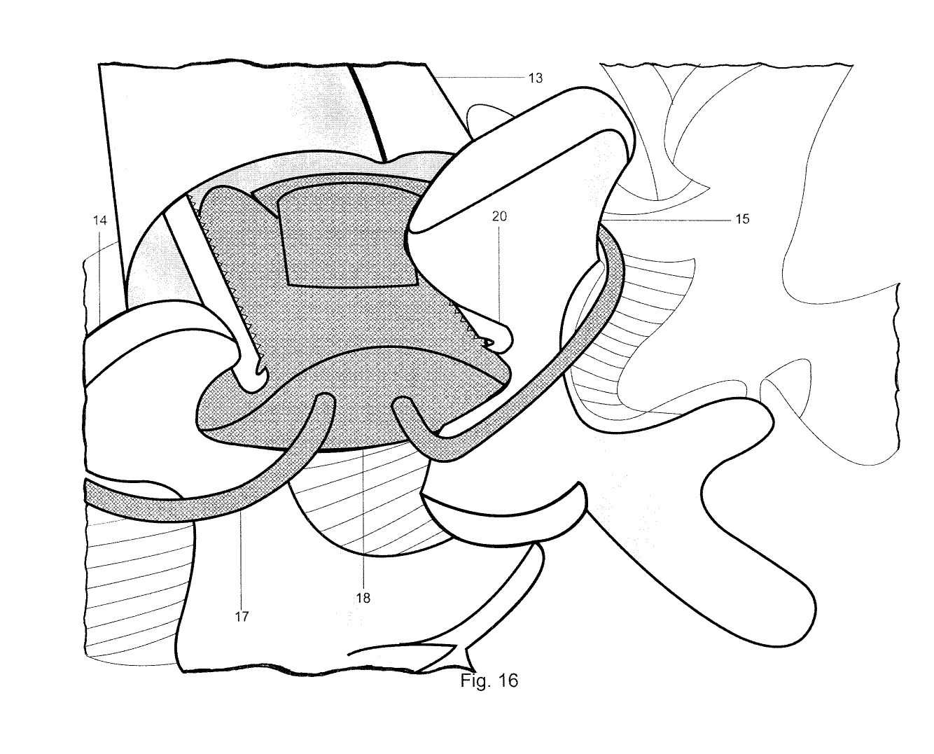

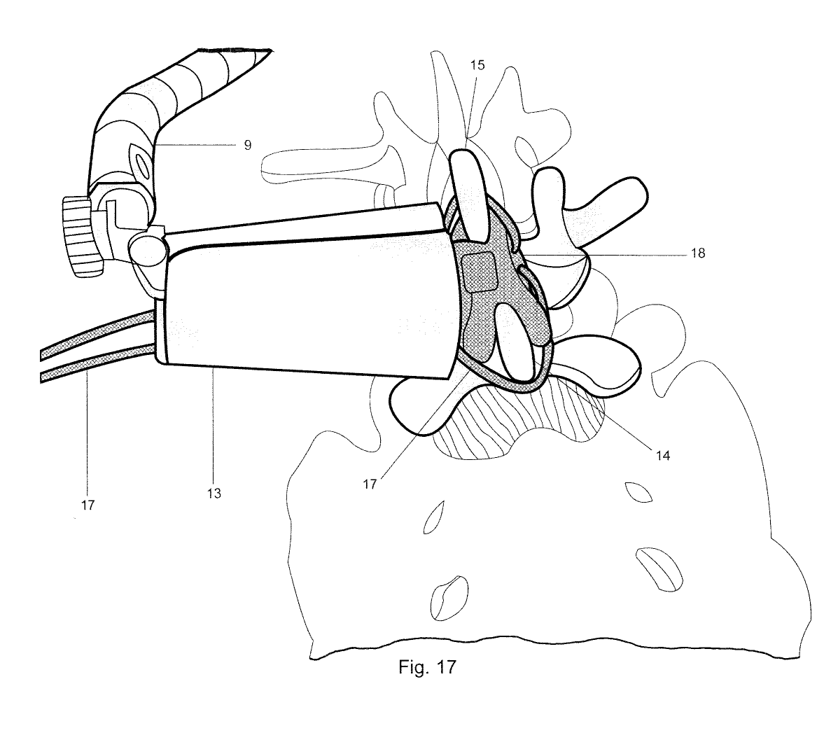

Regarding FIGS. 16 and 17, the implant DIAM® (18) is shown, once it is free from the pressure and deformation of clamps (20), placed in the corresponding interspinous space and with the sutures surrounding the corresponding spinous apophysis.

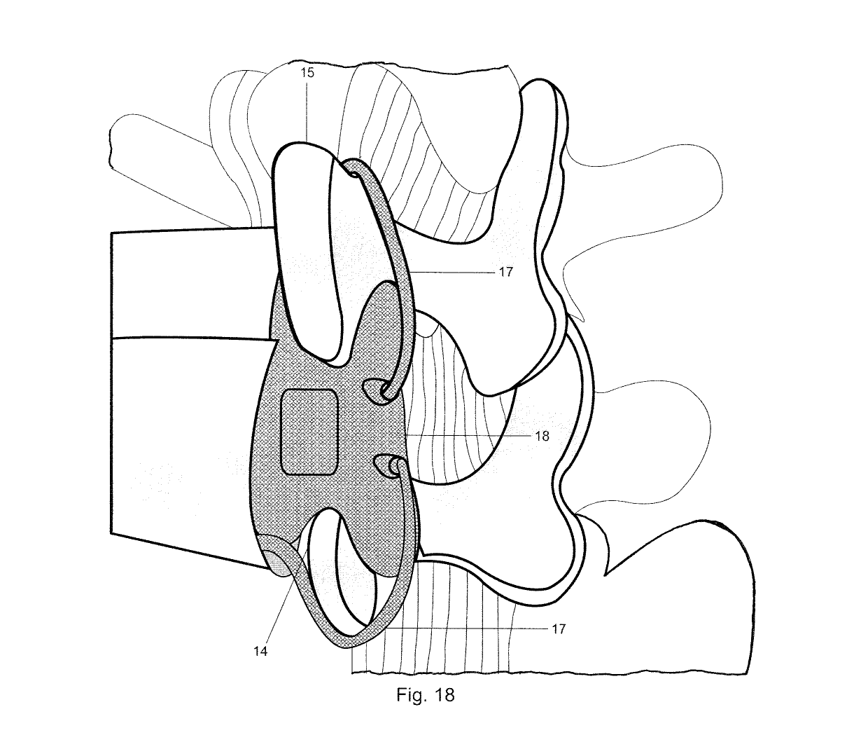

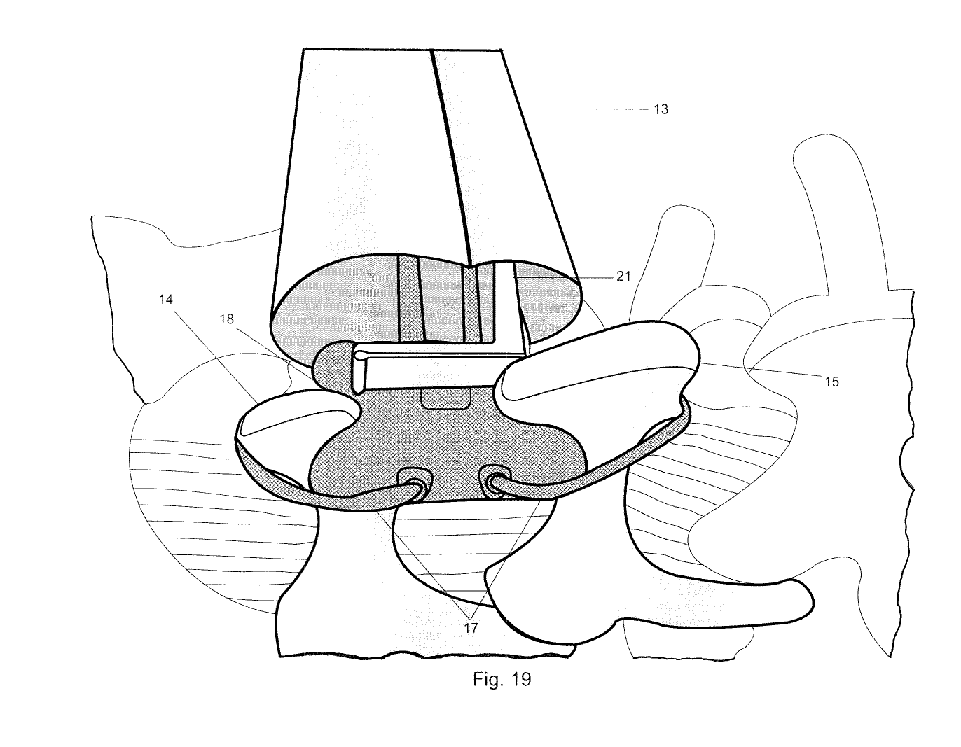

In FIG. 18 the sutures are fastened and the shape the implant DIAM® acquires is observed, which adapts around the shape of the spinous apophysis allowing a better fastening and stability at the moment of being submitted to the generated forces by the bones movement.

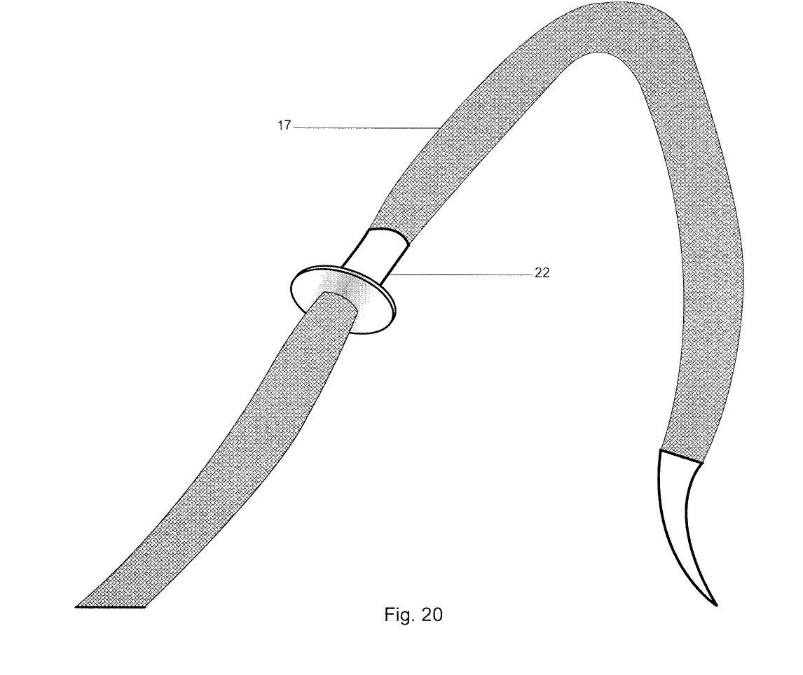

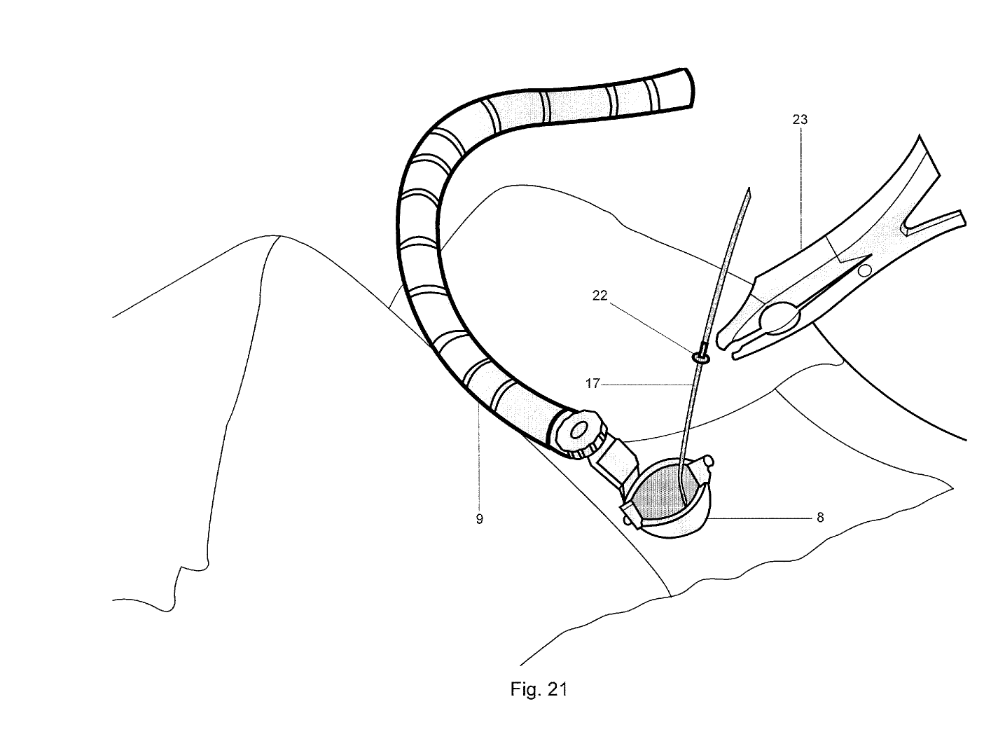

The fastening of the sutures is made by the aid of the clamp (21), which keeps the implant DIAM® (18) in a fixed position while the sutures are pulled as it is shown in FIG. 19, to fasten such implant and avoid it to move. In FIG. 20 the fastening of each suture through a bushing (22) is clearly seen, this is made of titanium or any other bio-compatible material, which slides through the suture where the suture and the buttonhole are and it gets in touch with the body of the device DIAM®. The bushing (22) is held by strapping clamps (23) which are shown in FIG. 21, and once it is placed and fastened in its right site, the clamps are pressed to deform the bushing and avoid the suture to get loose. The procedure is repeated with the suture of the other end and once the adjustment is carried out, the bushing is deformed (strapped) to fasten the implant.

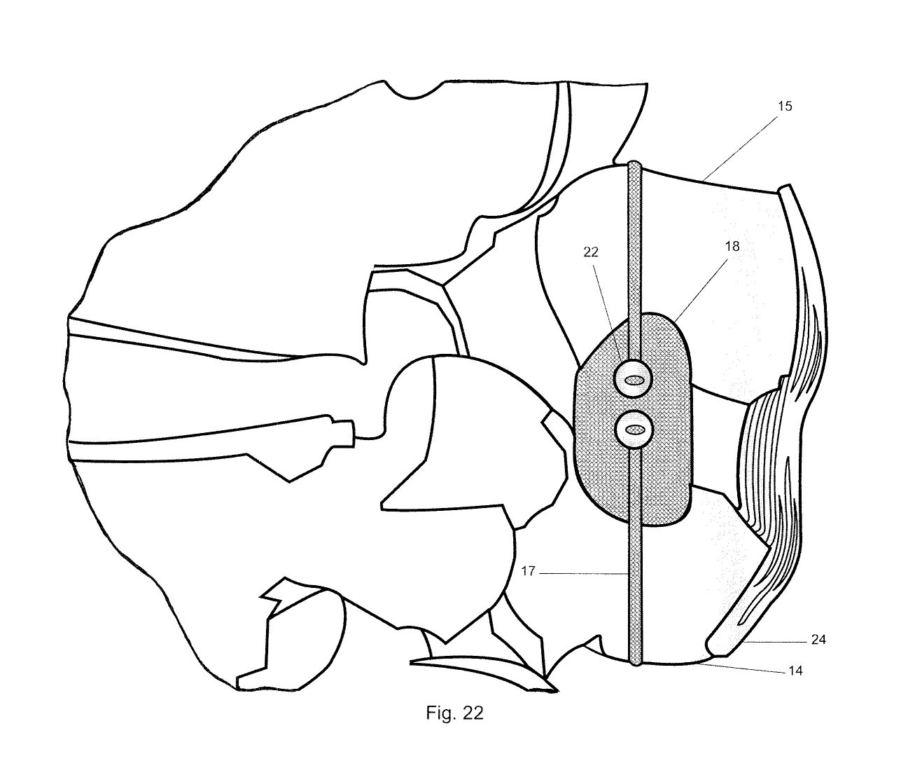

Regarding FIG. 22, a lateral view of the implant DIAM® (18) is shown, placed with the sutures (17) fastened through the bushings (22) in the interspinous space between the upper apophysis (15) and lower apophysis (14) and in which the interspinous ligament (24) is also observed.

Afterwards, the X-Tube® is removed and the common methodology of any micro endoscopic surgery continues.

In an embodiment of the disclosure, the anchoring of the implant DIAM® in L5-S1 may be performed without introducing an anchoring screw in S1, which simplifies in a significant way the procedures and risks of surgery.

If the patient presents spinous apophysis in S1, the method is carried out in the same way above mentioned.

In a particular case in which there is not spinous apophysis in S1, it is necessary to perform an additional step before the use of the hooks, which includes the use of an osteotome to carry out an osteotomy or slot on the base of the spinous apophysis of S1 that could vary from 1 to 4 mm of depth to allow the suture to fasten in such slot and not involving the nervous roots that are found deeper, afterwards the procedure follows the same stages of the previously disclosed method.

### Statistics

Statistics show a complication rate lower than 3%, mainly being fracture of spinous apophysis during the surgical procedure. However, there are some contradictions, since the implant DIAM® is mainly recommended in patients with degenerative disk disease, i.e. whose disk has suffered wearing away or, in some cases, disk hernias. Implants DIAM® are not recommended for patients with significant overweight, previous surgery, spondilolysthesis or deviation of spine (scoliosis). For a better understanding, in the following example the satisfying statistics of the use of the implant DIAM® are observed:

Example
50 patients with hernia of one or two disks
Women 13 (average 43.8 years old)
Men 37 (average 43.4 years old)
Levels:
L2-L3 1
L3-L4 1 (Wallis L4-L5)
L4-L5 and L5-S1 4
L4-L5 34
Results
Excellent 48 patients
Good 1 (overweight)
Regular 1 (Osteoporosis)
Times:
Surgery (one side) 1 level 40 minutes
   2 levels 60 minutes
Hospitalization:
   8 hours (39 patients, it means 78%)
   1 day (8 patients, it means 16%)
   2 days (3 patients, it means 6%)
Pain after surgery (1-3 days):
   Light 35
   Medium 8
   High 7
Back to work 10 days 40
   15 days 3
   Retired 7
After three months:
   46 patients (excellent result without pain)
   2 patients (good results, light pain)
   2 patients abandoned the treatment.
After 9 months:
   45 patients (remain excellent)
   2 patients (remain with good results)
   3 patients abandoned the treatment.
After 18 months:
   45 patients (remain excellent)
   2 patients (remain with good results)
   3 patients abandoned the treatment.

It is evident that many modifications and variations of the disclosure may be carried out as it is disclosed herein without departing from the spirit and scope of the same and, therefore, these limitations will be only imposed as established by the attached claims.

1. Instrumental of hook for its use in a method for minimally invasive spine surgery in interspinous space through a micro endoscopic surgery, comprising:
a retention sleeve disposed in a longitudinal and fixed manner in the upper part of the hook;
an enlarged rod with a curve-shaped end;
wherein the curve-shaped end terminates with a fishhook-shaped tip.

2. The instrumental of hook according to claim 1, wherein the curve-shaped end of such hook has a curvature range of 60±15 degrees regarding the axis of the rod and an arch length of 50±10 mm.

3. The instrumental of hook according to claim 2, wherein the external surface of the retention sleeve of such hook presents a knurled to provide an non-skid surface.

4. A method for a spine surgery in minimally invasive interspinous space through a micro endoscopic surgery, comprising:
locating the area of the affected central intervertebral space indentifying the upper spinous apophysis and the lower spinous apophysis and marking the insertion with a parallel line to about 30 mm from the medium line of the spine;
marking the area of insertion with a Steinmann nail;
carrying out an incision on the skin of about 24 to 26 mm;
introducing the dilators from the smallest to the largest diameter through the incision until reaching the largest diameter by which the controlled opening tube X-tube® of Medtronic® is introduced in its non expanded configuration;
expanding and addressing the tube towards the central interspinous space, where the implant positioning will be made.
fixing the X-Tube® with the fixation arm;
dissecting the front part of the interspinous ligament in the central interspinous space respecting the upper part to make the excursion of the suture easier through the use of a curve Kerrison clamp, preserving the supraspinous ligament;
creating a passing channel with the chisels for the instrumental of hook and the sutures around the spinous apophysis taking part in the operation;
measuring the space with the measurer to determine the size of the DIAM that will be employed using Peang clamps or some sharp clamps as distracters;
addressing the X-Tube® towards the upper spinal apophysis and introducing the proper hook depending on the size of the spinous apophysis, which must reach the central interspinous space to achieve retention of the sutures, and making such hook pass from the upper interspinous space through the channel created previously by using the chisels until the central interspinous space where the implant will be placed, leaving the fishhook-shaped tip of said hook towards the front of the tube;
anchoring the suture of the implant in said fishhook-shaped tip, passing it at the back of the upper spinous apophysis and taking it out on the front; and repeating the X-Tube addressing and suture anchoring steps now from the lower interspinous space, surrounding the lower spinous apophysis until the central interspinous space where the implant will stay;
once the two sutures are out of the tube, the upper suture passes through the upper buttonhole from top to bottom and the lower suture passes through the lower buttonhole from bottom to top;
fitting with the two sutures towards the front of the tube;
fastening and deforming the implant laterally in the direction of the sutures with the aid of Peang clamps;
introducing the implant within the X-Tube® to the central interspinous space in a slippery way while sutures are being fitted by applying tension in the ends of such sutures,
freeing the pressure of such clamps when the implant is in the central interspinous space;
fitting the sutures to the body of the implant and pressing by using the mallet to avoid movement while sutures are pulled and fitted;
fastening the sutures once they are fixed by using a strapping clamp which fastens the bushing that slides through each of the sutures to the base of the suture and the buttonhole, and deforming (strap) such bushings by pressing the clamps to avoid the suture to move, repeating for the other suture;
cutting the sutures close to the bushing as much as possible; and
removing the X-tube and finishing with the established protocol for micro endoscopy surgery.

5. The method according to claim 4, wherein the instrumental of hook goes though the channel created by the chisels around the upper spinous apophysis and around the lower spinous apophysis to the central intervertebral space to allow the fishhook-shaped tip to remain uncover in such central intervertebral space.

6. The method according to claim 4, wherein the additional step is carried out to perform an osteotomy or slot in the base of the spinous apophysis of S1 of about 3 to 4 mm of depth to allow the suture to be fastened in that slot, if there is not a spinous apophysis in S1.

7. The method according to claim 4, wherein the steps of addressing the X-Tube and anchoring the suture may be performed first in the upper spinous apophysis or in the lower spinous apophysis indistinctly.

8. The method according to claim 4, wherein the bushing may be made of titanium or any other bio-compatible material.
